(19) United States
(12) Patent Application Publication
(10) Pub. No.: US 2010/0279391 A1
(43) Pub. Date: Nov. 4, 2010

(54) METHOD AND SYSTEM FOR REMOVING CO2 FROM THE ATMOSPHERE

(76) Inventors: Walter Steven Rosenbaum, Paris (FR); Jörn Bach, Konstanz (DE)

Correspondence Address: DICKINSON WRIGHT PLLC, 1875 Eye Street, NW, Suite 1200, WASHINGTON, DC, 20006, US

(21) Appl. No.: 12/777,966

(22) Filed: May 11, 2010

Related U.S. Application Data
(63) Continuation-in-part of application No. 12/693,965, filed on Jan. 26, 2010.
(60) Provisional application No. 61/147,317, filed on Jan. 26, 2009.
(60) Provisional application No. 61/175,253, filed on May 4, 2009.
(60) Provisional application No. 61/223,949, filed on Jul. 8, 2009.

(51) Int. Cl. A62D 3/02; C12M 1/00

(52) U.S. Cl. 435/262.5; 435/289.1

(57) ABSTRACT

A system for removing gaseous pollutants from air enriched with the pollutants includes a cultivation environment configured to cultivate vegetation under exposure to pollutant enriched air, and a transformation unit configured to transform an encapsulation material to a transitory viscous or liquid state. Further, the system includes a mixing unit configured to combine the vegetation with the viscous or liquid encapsulation material, and a receptacle configured to receive a mixture of vegetation and viscous or liquid encapsulation material, wherein the mixture hardens within the receptacle.

### CROSS-REFERENCE TO RELATED APPLICATIONS

This application is a continuation-in-part of Non-provisional application Ser. No. 12/693,965, filed Jan. 26, 2010, which claims the benefit of priority to Provisional application Ser. Nos. 61/147,317, filed Jan. 26, 2009; 61/175,253, filed May 4, 2009; and 61/223,949, filed Jul. 8, 2009, the contents of which are hereby incorporated by reference.

### BACKGROUND OF THE INVENTION

The various embodiments described herein generally relate to removing gaseous pollutants from the atmosphere. More particularly, these embodiments relate to a method and system for removing CO₂ from the atmosphere.

Our current society, environment and economy are integrally tied to a carbon based energy cycle. Although nuclear and renewable energy are expected to make increasing contributions to world-wide energy needs, the major contribution in any foreseeable time frame will come from fossil fuel, predominantly coal, which adds carbon dioxide, hereafter referred to as CO₂, and other pollutants directly into the atmosphere. At present nearly 60% of the electrical power generation is affected via coal and almost 90% of electricity comes from some fossil fuel base. Although it is known to filter out some of the particulate matter that contributes to, for example, acid rain, the main byproduct of combustion, CO₂, is not presently containable in a way that the pollutants are permanently prevented from re-entering or being released back to the atmosphere.

The magnitude of the CO₂ problem becomes more apparent when it is considered that a 500 megawatt coal powered generating station contributes approximately 2,5 Mio tons of CO₂ yearly into the atmosphere. There are about 600 such stations in operation in the US alone and about 5 times that number in 24/7 operation world-wide.

Nature attempts to handle the deluge of CO₂ by photosynthesis and oceanographic geological formations of CO₂based sedimentations such as limestone. In essence, nature attempts to keep a CO₂ balance by means of long term sequestering or re-capture in non-gaseous states. The permanence of photosynthesis sequestering such as via woodlands is relatively short compared to CO₂ sequestered in limestone.

Most currently artificial means of sequestering have been under investigation such as pumping CO₂ in gaseous form into underground natural geological formations. The shortcomings of such an approach is that suitable underground sequestering sites are rare and, hence, can not possibly be co-located with all the CO₂ intensive sites that require them, and, more importantly, the ability to contain almost indefinitely massive amounts of CO₂, under tremendous pressure, that will not leak to the surface or rupture due to shifts in the earths crust is problematic at best. Alternatives to underground sequestering are equally tenuous such as schemes entailing pumping CO₂ into deep ocean where thermal-clines are proposed as a means to secure capture via inversion layers concerning long term ocean conditions. Such mass storage approaches also have the immediate health risk attendant with the potential for massive releases of CO₂ back into the environment with regional in concentrations that are dangerous or even toxic to human life.

Other approaches such as that now being researched by Calera attempts to put CO₂ into chemically inert form such as results by intermixing seawater and CO₂ to create a precipitant of calcium carbonate and magnesium carbonate, that can in theory be used as one of the constituents of cement. In practice the process produces an acidic byproduct and questions of scalability to an industrial scale remain (New York Times, Mar. 21 2010).

Other approaches such as that of David Bayless of the Ohio University Coal Research Center have focused on developing bioreactors that absorb carbon dioxide emissions from fossil fuels with the help of heat-loving algae. The resulting algae, however, are used for bio-fuel or animal feed which means their sequestering is very short before they reenter the environment in a gaseous CO2 state.

Hence a need exists for a means of sequestering of CO₂ that is for all practical purposes permanent and by its nature independent of geology. A further need exists that said sequestering be of virtually unlimited in its duration. Another requirement is that the mode of sequestering be impervious to release by shifting of the earth's crust or deep sea currents and conditions. A further requirement is that any conceivable leakage be both environmentally and human health-wise inconsequential. It is further desirable that the sequestering be able to encapsulate other pollutants such as nitrates, nitrites or heavy metals. Additionally, it is further desirable that the sequestering can be done at any location independent of geology and be the concatenation of low-technology processing steps. Lastly, it would complete an ecologically positive sequestering process if the sequestering medium was another industrial and societal waste product. Permanent and secure storage of CO₂ and other pollutants is not guaranteed when it is used in its original form. Therefore such storage would be greatly facilitated if, prior to storage, the pollutants would be “channeled” through procedures that effectively destroy them by affecting the molecular structure.

A common naturally occurring example for such procedures is photosynthesis, where the CO₂ molecule is used together with H₂O using sunlight as an energy source to synthesize CH₂O. Numerous projects exist that aim at exploiting this capability, especially of algae, to filter and process CO₂ from the exhaust fumes of power plants. These fumes are directed into the water of so called racing ponds or bio reactors. The algae in those facilities filter the CO₂ enriched water and incorporate it into their own bio mass. Reports such as http://www.jacobs-university.de/news/iubnews/15585/index.php.de are that up to 2 tons of CO₂ can be incorporated into 1 ton of algae dry mass.

However, with respect to the permanent removal of CO₂ from the atmosphere or from exhaust fumes of industrial plants this method is definitely insufficient since the CO₂ is, under normal circumstances, only “parked” within the biomass, which is usually intended for the production of animal food or for the generation of so called bio diesel. In both cases the CO₂ bound within the biomass is sooner or later returned to the atmosphere either by natural oxidation, burning as fuel or otherwise or by the digestion processes of animals. The same happens in naturally occurring deterioration commonly referred to as “rotting”, through the activity of bacteria. Neng Zing (http://www.cbmjournal.com/content/3/1/1) reports the suggestion for CO₂ sequestering via wood burial that involves dead trees being buried underground to prevent them from rotting.

To fully exploit the CO2 consuming potential of such procedures, of which plant or algae growth is only an example, it is therefore necessary that the end product of the CO₂ consuming procedures is neither burned nor used as food material or stored in its original faun but isolated from deteriorating environmental influences such as bacteria.

### SUMMARY OF THE INVENTION

The object of this invention is to provide a system and method that permanently removes pollutants, especially CO₂, from pollutant enriched air, such as power plant or other industrial exhaust fumes, but also from the general atmosphere or other polluted environments, in a way that said pollutants are permanently prevented from re-entering the atmosphere.

The first object of the disclosed invention is to bring the CO₂ into a form, in which it is neutral and no longer immediately harmful to the environment and also has a much more economic storage volume.

This is achieved by employing the photosynthetic capabilities of certain plants, preferably water based algae. These plants consume CO₂, and, using light, preferably sunlight and water (H₂O) synthesize CH₂O releasing O₂ as a waste product.

In the first phase of the disclosed method this procedure is used to neutralize the CO₂ by directing the CO₂ enriched air into the aquatic cultivation environment of algae, where the CO₂ is consumed by the algae and converted to CH₂O to aid in building bio mass.

This processional step is already known for the production of bio diesel from algae using power plant exhausts. However, since these installations are concerned with the production of certain goods the cultivation of algae has to take place in extremely well controlled environments and under carefully monitored light and temperature conditions to ensure that a high purity of a certain strain of algae is achieved.

The method disclosed in this invention, on the other hand, is only concerned with achieving maximum of CO₂ absorption of the algae.

Since this storage form is impermanent and prone to decomposition, another object of the invention is to encapsulate this storage form in a way that prevents it from contact with air and microorganisms and thus decomposition.

This is achieved by first sterilizing the algae to remove decomposing agents such as bacteria and microorganisms and then by mixing or combining it with a non-biodegradable material such as a polymer to isolate it from further contact with the air or decomposing agents.

As a further protection against damage or decomposition by environmental influences this primary encapsulation is supplemented by a secondary encapsulation that surrounds the mixture with an additional layer of protection.

The units that are the final result of this procedure can be molded in a way that facilitates transport and storage.

The system and method disclosed in this invention have the advantage to transform the gaseous CO₂, which is toxic in high concentration and is considered to be responsible for the greenhouse effect into easily manageable storage units.

These units can be very compact, have a highly economic storage volume of up to 2 t of CO₂ per ton of algae, and do not pose the same danger of leaking the CO₂ back into the environment, or noxious byproducts, as in other forms of CO₂ storage, such of sequestering in rock fissures or deep sea sequestering or retention by chemical interaction.

### BRIEF DESCRIPTION OF THE SEVERAL VIEWS OF THE DRAWINGS

The novel features and method steps characteristic of the invention are set out in the claims below. The invention itself, however, as well as other features and advantages thereof, are best understood by reference to the detailed description, which follows, when read in conjunction with the accompanying drawings, wherein:

FIG. 1 shows an illustration of one embodiment of a system for removing gaseous pollutants from the atmosphere; and

FIG. 2 is a flow chart of one embodiment of a method of removing gaseous pollutants from the atmosphere.

### DETAILED DESCRIPTION OF PRESENTLY PREFERRED EMBODIMENTS

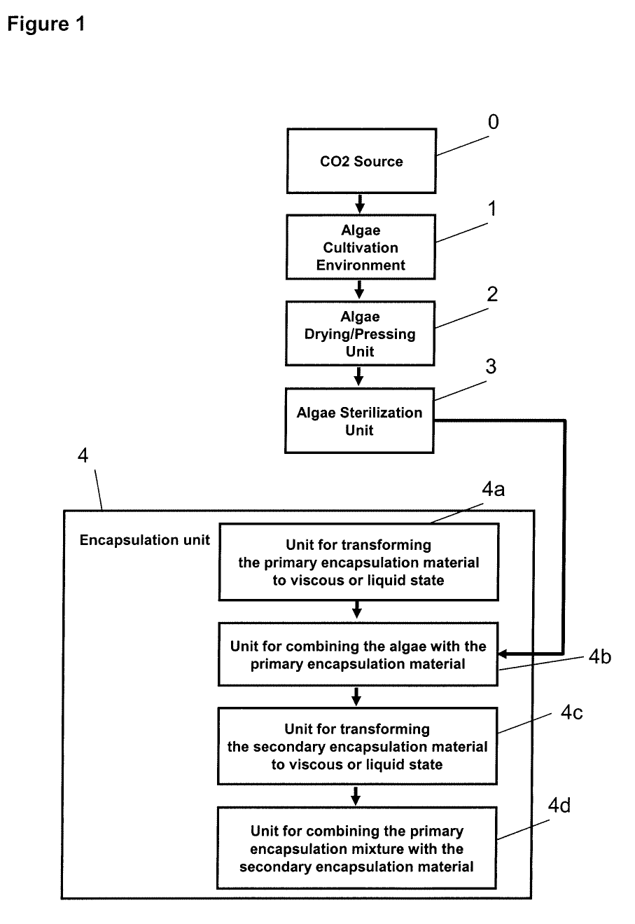

FIG. 1 shows an illustration of one embodiment of a system for removing pollutants from the atmosphere. One example of a gaseous pollutant is CO₂. It is contemplated that other pollutants, liquid or metal, which may be consumed by plants that pollutants that perform oxygenic photosynthesis, may be removed as well. Briefly, the system neutralizes the pollutant via consumption by algae or other vegetation that performs oxygenic photosynthesis and are able to consume pollutants, and, then, prevents a resulting bio mass from CO₂-generating decomposition by combining it with a multi layer encapsulation.

The illustrated system includes an algae cultivation environment 1, which receives CO₂ enriched air from a CO₂ source 0, an algae drying or pressing unit 2, an algae sterilization unit 3 and an encapsulation unit 4. The encapsulation unit 4 has several (sub)units: a unit 4a transforms a primary encapsulation material into a temporary viscous or liquid state, a unit 4b combines the algae with the primary encapsulation material to create a primary encapsulation, a unit 4c transforms the primary encapsulation material into a temporary viscous or liquid state , and a unit 4d combines a secondary encapsulation material with the primary encapsulation to create a secondary encapsulation. Obviously, many modifications and variations of the present invention are possible in light of the above teachings. It is, therefore, to be understood that within the scope of the appended claims, the invention may be practiced otherwise than as specifically described.

The CO₂ source 0 is an electrical power plant, any other industrial facility or a CO₂ storage unit. In one embodiment, the CO₂ source 0 is the atmosphere.

The algae cultivation environment 1 is an enclosure containing a predetermined cultivation medium. For algae, the cultivation medium is a variant of water, such as fresh water, brackish water or saline water according to cultivation requirements of the type of algae used. Since the photosynthesis process performed by the algae uses sunlight, or artificial light of a comparable light spectrum, as an energy source, the enclosure is constructed to allow light to reach the algae. In one embodiment, the cultivation environment 1 is configured for sunlight to reach the cultivation medium. The sunlight may be replaced by or supplemented with artificial light of the same wavelength spectrum. In one embodiment, the direction of light into the cultivation environment 1 can be controlled and optimized with an arrangement of mirrors or light guides, e.g., glass fibers.

The algae cultivation environment 1 is in one embodiment an open shallow pond, or a series of such ponds filled with water and open to sunlight with circulation provided by paddle wheels, so called raceway-type ponds. The introduction of CO₂ to this environment can be achieved by a system of perforated underwater tubes that distribute the CO₂ by releasing it in streams of bubbles. In another embodiment the open shallow ponds may be used without circulation.

In one embodiment, the cultivation environment 1 may be a narrow tank, or a series of narrow tanks filled with water and made from a material transparent to light, so called bio reactors. Such installations are known for the cultivation of algae, e.g., for the production of bio diesel. It is contemplated that the cultivation environment 1 may be selected from a variety of containers filled with water and transparent to light. Further, it is contemplated that in an artificial cultivation environment 1 the distribution of the CO₂ can be achieved by a system of perforated underwater tubes that distribute the CO₂ by releasing it in streams of bubbles.

The cultivation environment 1 is not necessarily artificial and the algae growth may not be the result of intentional cultivation. Such environments can be natural ponds or other bodies of water, artificial or natural, that are polluted by uncontrolled algae growth. Since algae blooms pose a major problem due to the washing of fertilizers from agriculture into the water the method described herein can also be applied to permanently remove such biomass. As stated above, the term “algae” is a representative of any kind of vegetation capable of performing oxygenic photosynthesis.

The algae biomass, once removed from the cultivation environment 1, is subject to a drying procedure in the drying/pressing unit. 2. The function of this unit is to remove water and excess moisture from the biomass. This can be accomplished by pressing and subsequent drying or by pressing or drying alone. The drying/pressing unit 2 in the preferred embodiment is a mechanical press operated by hydraulics or some other means of mechanical power transmission. In another embodiment the drying facility may contain a centrifuge or similar device to remove water from the biomass by means of rapid rotary movement.

In one embodiment the algae is not dried at all, or only partially dried before being subjected to further processing. The drying unit 2 is an open place where the drying is accomplished by solar heat. In another embodiment the drying unit 2 is a source of heat, or a source of microwaves. The heat applied in this case may be just sufficient to remove moisture from the bio mass but it may also be high enough to turn part or all of the bio mass into so called bio char, which is basically a form of charcoal. The last option would require that the heating facility could operate in a vacuum. Such facilities are well known for bio char production.

The drying/pressing unit 2 can also be formed of more than one of the above facilities allowing sequential pressing and drying procedures. It may also be that the bio mass arrives at the drying/pressing unit 2 in a dried state. In this case the drying/pressing unit 2 may only function as a pressing unit, pressing the bio mass into compact shapes.

In the sterilization unit 3 the algae is subjected to a sterilization procedure to arrest the biological decomposition of the biomass. In one embodiment, the sterilization unit 3 is a source for ionizing radiation; either high-energy electrons or X-rays from accelerators, or by gamma rays (emitted from radioactive sources as Cobalt—60 or Cesium—137). Since high-energy electrons such as X-ray can only penetrate organic material up to a depth of about 3 cm, the algae layer treated in this way has a thickness that does not exceed that thickness. If electron rays are applied from above and below the thickness of the layer may be up to 6 cm. Gamma radiation can irradiate any practical depth of algae given the nominal dosage of 1.12 to 1.33 MeV (Cobalt—60) and 0.66 MeV (Caesium—137) as is the practice for food and hospital sterilization.

In one embodiment, the sterilization unit 3 is a source of ultraviolet radiation at a wavelength of 240-280 nm, or a source of micro wave radiation. In another embodiment, the sterilization unit 3 is a source of heat, such as, for example, an autoclave, and/or a source of hot steam, optionally in combination with a pressure of at least 70,000 pounds per square inch. Further, the sterilization unit 3 may include a chemical treatment feature, using substances such as ethylene oxide gas, ozone, chlorine bleach, ortho—phthalaldehyde, hydrogen peroxide, peracetic acid, or other appropriate substances. In another embodiment the sterilization unit 3 may be implemented in the unit 4-b that combines algae and the primary encapsulation material, since the melting temperature of about 250° C.-300° C. for some encapsulation materials may be sufficient to achieve a high sterility assurance level.

In the encapsulation unit 4 the algae is combined with the primary encapsulation material. This encapsulation requires that the primary encapsulation material is transformed into a temporary or transitional viscous or liquid state. How this is achieved depends on the kind of encapsulation material used.

The unit 4a transforms the primary encapsulation material into a temporary viscous or liquid state and is a source of heat to melt the material and a vat to contain the material in its molten state. This approach is primarily used with materials like polymers, glass, cement or metal. The temperatures required for the different encapsulation materials are detailed below. In another embodiment the unit 4a to transform the primary encapsulation material into a temporary viscous or liquid state is configured to mix the material with water or another solvent.

While the primary encapsulation material is in its temporary or transitional viscous or liquid state it is combined in unit 4b with the algae. The unit 4b is a vat constructed of a material that has a melting point higher than the melting point of the primary encapsulation material.

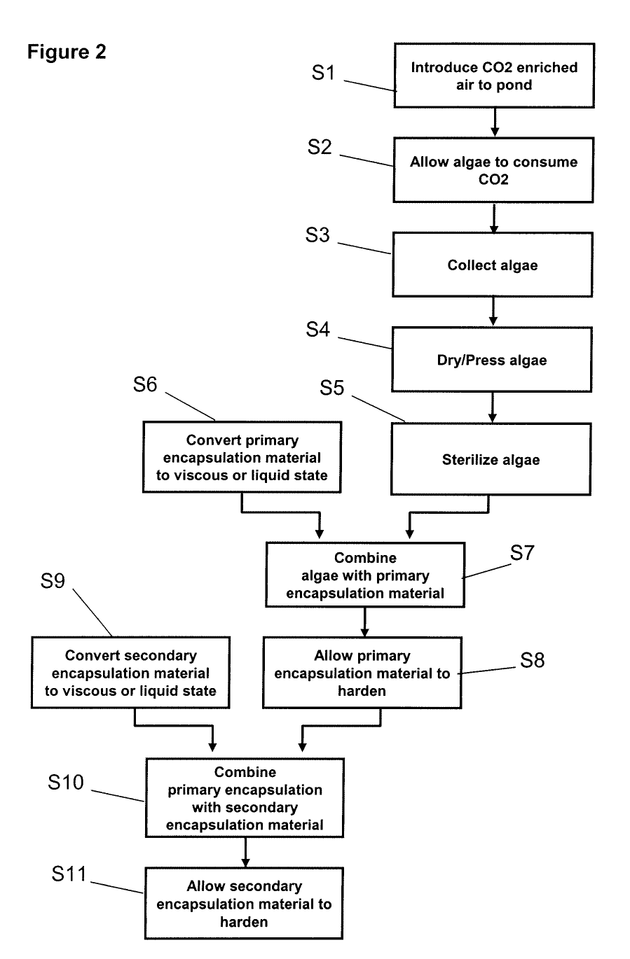

FIG. 2 is a flow chart of one embodiment of a method of removing gaseous pollutants from the atmosphere as performed in the system illustrated in FIG. 1. In a step S1, CO₂ enriched air from the CO₂ source 0 is introduced to the algae cultivation environment 1. In one embodiment CO₂ enriched exhaust fumes from power plants, synthetic fuels plants, liquid fuels refineries, cement kilns, ammonia production facilities, or other industrial facilities are directed to the algae in the cultivation environment 1 via a system of perforated tubes that run beneath the water's surface and release the CO₂.

In another embodiment the method is applied to CO₂ coming from CO₂ storage facilities. These can either be temporary storage facilities such as tanks or pipelines, but also what is commonly called carbon containment and storage (CCS) facilities intended for otherwise long-term storage where pressure buildup can be bled off into the algae cultivation environment 1, or such CSS where such long-term storage is in threat due to having become damaged and are leaking CO₂ into the atmosphere or groundwater.

Alternatively, CO₂ enriched air can be directly taken from CO₂ enriched atmosphere and pumped into the cultivation environment. In another embodiment CO₂ can be directly taken from the air to create a CO₂ enriched atmosphere and pumped into the cultivation environment. Another embodiment deals with cases were CO₂ has not been artificially infused into the algae cultivation environment, but has been introduced by natural procedures such as atmospheric interaction the surface of ponds, flowing water etc.

In a step S2, the CO₂ is consumed by the algae in the cultivation environment 1. The cultivation environment 1 is filled with a cultivation medium for algae, which is water according to the cultivation requirements of the type of algae used, such as freshwater, brackish water or saltwater. The term bio mass or algae as used in this disclosure refers to vegetation that is capable to consume CO₂ and process it using sunlight and H₂O in a chemical procedure known as oxygenic photosynthesis. It is to be considered synonymous with any kind of vegetation or microorganism capable of this procedure. Other kinds of microorganisms may also be employed such as, for example, Acidianus ambivalens. This process converts the CO₂ using H₂O into CH₂O, and releases oxygen. Since oxygenic photosynthesis is a process found in all green plants, algae and cyanobacteria all these substances may be used for this purpose.

In one embodiment the CO₂ consumption is achieved via algae, for example, of the type Chlorella. In another embodiment waste products and residuals of other procedures involving algae or other types of bio mass are used. Such procedures can be, for example, the production of bio diesel, cosmetics or other products, where a residual remains, after the algae have been processed for their oil. In this embodiment the managed growth procedure is not necessary. Another embodiment employs algae that are not cultivated but grow naturally.

In another embodiment, land-based plants or parts thereof are used for CO₂ consumption, or digestive products of organisms that have consumed the plant material described above as well as substances generated by processing the algae via chemical or technological means such as, but not limited to, changes in temperature and pressure. The term “algae” used subsequently also extend to such secondary or ternary etc. algae products.

In a step S3, the algae are harvested or collected from the cultivation environment 1. Such harvesting or collecting methods are known from cultivation of algae for food and non-food products. In one embodiment the algae are harvested from its aquatic environment by mechanical filtration, or by so called bio-flocculation or auto-flocculation triggered by engineered changes to environmental conditions of the growth media or by addition of flocculation-stimulating compounds or materials, by mechanical filtration.

In one embodiment indirect harvesting by biological concentration is applied. An example for this procedure is to have certain types of fish such a Tilapia fish feed on the algae and then collect their droppings. In another embodiment hydrophobic algae species are cultivated that naturally layer themselves upon removable surfaces. In another embodiment algae strains with hydrophobic surfaces are cultivated that may be harvested by circulating solids with similarly hydrophobic surfaces through the growth media, onto which the algae may accumulate. In systems that cultivate algae as a bio-film on solid surfaces, algae may be removed from the surfaces for subsequent encapsulation, or for production of multiple co-products including encapsulation, or encapsulation may encompass the solid surfaces on which algae is cultivated, as in layered plastic-algae-plastic sheets described below.

In another embodiment a combination of two or more of the above methods is used. In a preferred embodiment the algae, after being harvested, are subjected to further processing in their original state.

In one embodiment the algae are subjected to some form of shredding or grinding. This may be necessary if the biomass does not consist of micro algae but of larger macro-algae or of plants whose size makes them awkward to handle for the subsequent processing stages. This shredding procedure can take place at any processing stage prior to encapsulation.

In a preferred embodiment the algae, once removed from its growing environment is subjected in a step S4 to a drying procedure in the drying/pressing unit 2. The drying can be achieved by processing the algae with devices that apply mechanical pressure to press the water out of the biomass. Such devices can be, for example, heavy rollers or a hydraulic press. As a result of this procedure the algae may also be pressed and molded into compact shapes, such as blocks. Another method to achieve drying is centrifugation of the biomass.

The drying can be accomplished by subjecting the algae to (artificial) heat. This may be achieved in a drying installation that has an internal drying chamber with controllable temperature and may work either by heating up one or more of the internal surfaces of this drying chamber or by circulation of hot air. Such devices are known. A drying chamber may also operate using microwaves, or the drying process may be performed in a large drying hall.

The heat and or pressure applied or generated may be just sufficient to remove moisture from the bio mass but it may also be high enough to turn the bio mass partly or fully into so called bio char, which is basically a form of charcoal. The last option would require that the heating facility could operate in a vacuum that may also be pressurized. In another embodiment the biomass can be dried by freeze drying.

In another embodiment the drying of the algae may also be achieved by spreading it on an open surface and use the warmth provided by the sun to accomplish the drying.

Also the drying can be achieved by a combination of the above procedures such as first pressing the water from the biomass by means of mechanical pressure and subsequently removing residual moisture via the application of heat. Molding of the biomass into compact blocks can also be achieved by mechanical pressure after the water and residual moisture have been removed by other methods.

It may also be that the bio mass arrives at the drying/pressing unit 2 in a dried state. In this case the drying/pressing unit 2 may only function as a pressing unit, pressing the bio mass into compact shapes. Also, during or prior to pressing additional substances may be added to the bio mass that serve as binders or stabilizers, or CO2 absorbers or anti bacterial agents. Further, the algae may not be dried at all or only partially dried before being subjected to further processing.

Also other and additional forms of processing and use of the algae prior to encapsulation are possible and still within the scope of this patent. As an example the bio char optionally produced in step S4 may prior to encapsulation be used to filter impurities from water.

In the illustrated embodiment, the algae are sterilized in a S5 in the sterilization unit 3 to kill any bacteria and microorganisms that may cause or facilitate algae decomposition. This can be accomplished with methods known from food and non-food sterilization and may be applied prior, during or even after encapsulation. In one embodiment such sterilization is performed prior to encapsulation. The sterilization is accomplished by exposure of the algae to ionizing radiation; either high-energy electrons or X-rays from accelerators, or by gamma rays (emitted from radioactive sources such as Cobalt—60 or Cesium—137).

Since high-energy electrons such as X-ray can only penetrate organic material up to a depth of 3 cm, the algae layer treated in this does not exceed that thickness. If electron rays are applied from above and below the thickness of the layer may be up to 6 cm. Gamma radiation can irradiate any practical depth of algae given the nominal dosage of 1.12 to 1.33 MeV (Cobalt—60) and 0.66 MeV (Caesium—137) as is the practice for food and hospital sterilization.

Sterilization may be achieved by exposing the algae to ultraviolet radiation at a wavelength of 240-280 nm. In another embodiment sterilization may be achieved by subjecting the algae to micro wave radiation. In another embodiment sterilization of the algae can be achieved by heating treatment. In one embodiment this is accomplished by a method known as dry heat sterilization. In this method the algae is heated to at least 160° C. for two hours or 170° C. for one hour.

Further, the sterilization may be accomplished by one of the forms of moist heat sterilization that use hot air together with moisture to kill germs and bacteria. In one embodiment sterilization is accomplished by hot pressurized steam. Also, the algae are subjected to high pressure of at least 70,000 pounds per square inch. Under such conditions most, but not all, microorganisms are killed. In another embodiment sterilization can be achieved by chemical treatment as explained above.

Combinations of two or more of the above methods may be used to maximize the sterility assurance level (SAL). In one embodiment sterilization may be accomplished during primary encapsulation since the melting temperature of about 250° C.-300° C. for some encapsulation materials the primary encapsulation procedure may be sufficient to achieve a high sterility assurance level. Also all other forms of sterilization that arrest the biological decomposition of the biomass are still within the scope of this patent.

It may be determined that sterilization is unnecessary due to particular parameters or the general nature of subsequent encapsulation processes or long term storage systems. The electromagnetic radiation can be applied not only prior, but during or after primary or secondary encapsulation if the encapsulation materials have been used that can be penetrated by such radiation. In one embodiment combinations of two or more of the above methods are used prior to, during and after encapsulation to maximize the sterility assurance level.

The primary encapsulation procedure involves combining the algae with an encapsulating material or medium. This primary encapsulation surrounds the algae, either as a whole or in the form of smaller units, to prevent decomposition and subsequent generation of CO₂ or other greenhouse gases. Therefore, the primary encapsulation material is capable to isolate the algae from air, external moisture, microorganisms and any other agents that may lead to such decomposition. Also, the primary encapsulation procedure together with the subsequent secondary encapsulation procedure allows molding the resulting storage units in a way that facilitates transport and storage.

In a step S 6, the primary encapsulation material is transformed in the unit encapsulation unit 4a to a temporary viscous or liquid state. The details of this procedure depend on the type of encapsulation material used. In the preferred embodiment the encapsulation material is a polymer. Suitable polymers are those commonly produced by the plastic industry and found in abundance in collections of waste plastic:

PET (PETE), Plyethylene Terephtalate: Commonly found in soft drink bottles, water bottles, cooking oil bottles, peanut butter jars.

Substances generated by the polymerization of algae or other biological substances.

HDPE, high-density polyethylene: Commonly found in detergent bottles, milk jugs.

PVC, polyvinyl chloride: Commonly found on plastic pipes, outdoor furniture, siding, floor tiles, shower curtains, clamshell packaging.

LDPE, low-density polyethylene: Commonly found in dry cleaning bags, produce bags, trash can liners, and food storage containers.

PP, polypropylene: Commonly found in bottle caps, drinking straws, yoghurt containers, legos.

PS, polysterine: Commonly found in “packing peanuts”, cups, plastic tableware, meat trays, take-away food clamshell containers.

The above polymers related to the primary encapsulation become viscous at about 250° C.-300° C. In another embodiment, the encapsulation material is glass. For glass and the like the viscosity temperature is in the range of 800° C. In another embodiment such encapsulation material may result from reprocessing and rendering such waste polymer or glass from discarded industrial, private waste, landfills, ocean clusters, etc. The encapsulation material may further be a metal, substances of mineral origin such as clay or cement that can be mixed with water to achieve a viscous state and harden, geopolymers, including but not limited to those produced from industrial wastes, such as fly ash, olivine or other CO₂ absorber may be mixed into the encapsulation media.

A substance with a greater density and weight per unit than the encapsulation media may be intermixed with the encapsulation media to ensure the resulting encapsulation will sink to the bottom of a given aquatic storage area.

Any material that can be transformed into a temporary viscous or liquid stage by the application of a liquid solvent that allows it to be combined with the plant material and subsequently hardens may be used. Similarly, any material that can be transformed into a temporary viscous or liquid state that allows it to be combined with the plant material and subsequently hardens may be used. In one embodiment combinations of the two or more of the above materials are used concurrently.

In another embodiment encapsulation materials may be used that do not need to be liquefied because they already come in liquid form and are subsequently hardened by contact with air, changes in temperature or ultra-violets or other types of electromagnetic radiation or contact with other substances.

In a step S7, the primary encapsulation material, while still in the viscous or liquid state achieved in step S 6 is combined with the algae in encapsulation unit 4b. This is the primary encapsulation procedure. The primary encapsulation procedure takes place under conditions that prevent or minimize further contact with decomposing agents such as microorganisms. This may be accomplished in a sterile environment or an environment that is subjected to UV radiation. Alternatively, the sterility assurance level of the previously described sterilization procedures may be considered sufficient and encapsulation under sterile conditions may not be necessary.

The combination of algae and primary encapsulation material is achieved by transforming the primary encapsulation material temporarily to a liquid or viscous state in step S6 and then pouring the algae into the liquid or viscous primary encapsulation material, or pouring the liquid or viscous primary encapsulation material over the algae. Algae and encapsulation material may also be mixed or stirred by using additional mixing implements to achieve better distribution.

In another embodiment the primary encapsulation material is sprayed or poured over the algae. In another embodiment the primary encapsulation material may be applied by passing the algae through a bath of the encapsulant.

The temperature increase of the algae from mixing with the temporarily viscous or liquid encapsulation material, or from heat transfer immediately preceding such mixing, may result in partial gasification or pyrolysis of the algae that releases vapors, which may be captured for independent uses.

Additional substances may be introduced into this mixture for additional effects, such as material that is heavier than water, to allow underwater storage. Also, substances may be added that have CO₂ and other pollutant absorbing effects. A known substance is, for example, cement that is supplemented with magnesium oxide or olivine. Olivine or another CO₂ absorber may be mixed into the encapsulation media. Such substances may be also added to the unmixed bio mass at any convenient stage of the entire process.

In a step S8, the mixture of primary encapsulation material and algae is allowed to harden. To harden, the mixture may be poured in forms or molds, extruded into longitudinal forms, which can be subsequently cut to various lengths or otherwise manipulated, or left inside the mixing receptacle where the combining of algae and encapsulation material has taken place.

While in some embodiments only one layer of encapsulation may be sufficient to achieve isolation from decomposing influences, in a preferred embodiment the first layer of encapsulation is again surrounded by one or more layers of additional layers of encapsulating material. This is termed the secondary encapsulation. It is preferable that the secondary encapsulation is achieved using a material that is more permanent and more durable than the material used for primary encapsulation.

In a step S9, the secondary encapsulation material is transformed to a temporary viscous or liquid state in the encapsulation unit 4c. The details of this procedure depend on the type of encapsulation material used.

The secondary encapsulating layer is made of glass with a viscosity temperature in the range of 800° C. In another embodiment the secondary encapsulation material is a polymer, e.g., PET, substances generated by the polymerization of algae or other biological substances, HDPE, PVC, LDPE, PP, PS, or Polysterine, as mentioned above. Other materials are as described above with respect to the primary encapsulation.

In a step S10, the mixture generated in S 7 is combined with the secondary encapsulation material in encapsulation unit 4d. For secondary encapsulating materials that have a lower or significantly higher melting point than the primary encapsulation material, the secondary encapsulating material may be molten and than poured over the mixture of plant material and primary encapsulating material after the primary encapsulating material has solidified. This is done in a way so that the secondary encapsulating material is able to fully surround the solidified mixture of algae and primary encapsulating material. Similarly, secondary encapsulating materials that do not require melting may be poured over and around the mixture.

In step S7, the primary encapsulation material, while still in the viscous or liquid state achieved in step S6 is combined with the algae in Encapsulation Unit 4d. This is the primary encapsulation procedure.

For secondary encapsulating materials that have a higher melting point than the primary encapsulation material two approaches are possible. In one embodiment, the solidified mixture prior to being surrounded by secondary encapsulating material is surrounded by an in-between layer of an isolating material, such as clay, ceramics, grease or any other appropriate material that prevents the mixture from melting upon contact with the hot secondary encapsulating material. In another embodiment the sequence of operations is reversed as the secondary encapsulation layer is created first, as a kind of open container or receptacle, then the mixture of viscous primary encapsulation material and algae is poured into this container and the opening of the container is closed with a layer of secondary encapsulation material and sealed. In another embodiment the primary encapsulation may be passed through a bath of viscous or liquid secondary encapsulation material.

Additionally to the primary and secondary encapsulation methods already discussed other procedures are possible that are still within the scope of this invention. These procedures can be used for primary encapsulation, secondary encapsulation or both.

The encapsulation may be achieved by creating a layered composite whereby a layer of encapsulating material either preformed or still viscous is laid down and algae placed a top. Another layer of encapsulating material that is pre-formed or still viscous is sprayed or poured down on top of the previous layer of encapsulating material covered with algae.

In one embodiment the preformed plastic layer can also be the initial layer whereupon the algae are deposited. The layers of encapsulating material with algae are pressed together in a manner and environment intended to deplete or remove oxygen and create a hermitically sealed entity. The layered encapsulating material with sandwiched algae can be pressed by heated or other elements to further compartmentalize the encapsulated algae into small independently hermitically sealed nodules of encapsulating composite.

The layering can be individually encapsulated with secondary etc. encapsulation or the individual layering of encapsulating material and algae can be stacked and then in mass encapsulated by a secondary media etc.

The process of creating a layered encapsulating composite sandwiching plastic-algae-plastic can be performed using layers of pre-formed plastic sheet below and above the layer of plastic whereby the laminated composite is formed by evacuation atmosphere and under pressure and heat creating hermitically sealed nodules of encapsulated algae.

In another embodiment encapsulation can be achieved by ejecting the liquid or viscous encapsulating material by a process of extrusion whereby the encapsulating plastic in a viscous state is ejected via a mold-like orifice that may, for example, shape the encapsulating material into a tube-like extrusion.

The algae, either in its original form or pre-processed in the manner described above, can then be injected into the encapsulation material during the extrusion process to form, in the case of a tube like orifice, a spaghetti-like structure that is filled with the plant material intermixed with encapsulation material. This extrusion can be directly secondarily encapsulated with such material as described above or can then be transferred to a container or spooled before being encapsulated in any appropriate secondary encapsulation material discussed above.

While in the preferred embodiment the algae is combined with one or more layers of a temporarily liquid or viscous encapsulation material that is subsequently allowed to harden in another embodiment the method of encapsulation of said vegetative matter can be in containment cells, containers or other receptacles made of plastic or other materials and encapsulated in multiple layers of plastic and secondary encapsulating material.

In this embodiment any air may be evacuated to create a vacuum in the containment cell and its sealing done in a manner to maintain said vacuum. It is preferable in said embodiment that the algae or vegetative matter in said containment cell be sterilized as specified above and mixed with oxygen binding substances to complete the removal of any residual oxidants. The containment cell may also contain in addition to bio mass anti-microbe, anti-bacterial or antifungal substances. Such substances may also be included in all other encapsulation embodiments.

It is to be noted that the storage in containment cells may also be performed in a way that allows removal of the algae from the containment cell at a later time. In such cases encapsulation other than the containment container itself may not be applied. In another embodiment encapsulation is achieved by placing a layer of algae between two ready made sheets of plastic that are then vacuum sealed in a way that individual isolated nodules of algae are formed. It is contemplated that other substances and structures may be integrated into the secondary encapsulation for enhanced stability and durability.

The secondary encapsulation may be a ready-made durable sealable container that surrounds the primary encapsulation. The secondary encapsulation procedure can be repeated several times to form additional protective layers around the primary encapsulating material.

It is contemplated that the resulting storage units should be molded in a way that allows for easy transport and storage on land, underground or underwater.

In the preferred embodiment the entire procedure, including algae cultivation, sterilization, and encapsulation is performed in the same installation or in closely adjacent plants. In another embodiment various operational stages of the procedure can be separated and performed in different locations. For example the algae cultivation procedure can be performed in the immediate vicinity of a power plant and the algae could be transported to a different location for sterilization and encapsulation.

It is contemplated that other modifications and variations of the present invention are possible in light of the above teachings. It is, therefore, to be understood that within the scope of the appended claims, the invention may be practiced otherwise than as specifically described.

What is claimed is:

1. A system for removing gaseous pollutants from air enriched with the pollutants, comprising:
a cultivation environment configured to cultivate vegetation under exposure to pollutant enriched air;
a transformation unit configured to transform an encapsulation material to a transitory viscous or liquid state;
a mixing unit configured to combine the vegetation with the viscous or liquid encapsulation material; and
a receptacle configured to receive a mixture of vegetation and viscous or liquid encapsulation material, wherein the mixture hardens within the receptacle.

2. The system of claim 1, further comprising a source of light selected to irradiate the vegetation in the cultivation environment.

3. The system of claim 1, further comprising a system configured to guide air to the cultivation environment.

4. The system of claim 1, wherein the cultivation environment contains water as a cultivation medium.

5. The system of claim 1, where the vegetation is a species of algae.

6. The system of claim 1, further comprising a sterilization unit configured to sterilize the vegetation.

7. A method of removing gaseous pollutants from air enriched with the pollutants, comprising:
directing pollutant enriched air into a cultivation environment containing a vegetation;
transforming an encapsulating material to a transitory liquid or viscous state;
combining the vegetation with the liquid or viscous encapsulating material; and
transferring a mixture of vegetation and viscous or liquid encapsulation material to a receptacle and allowing the mixture to harden.

8. The method of claim 7, wherein the vegetation is algae.

9. The method of claim 7, wherein the encapsulation material is selected from the group consisting of polymer, glass, metal, and cement.

10. The method of claim 7, further comprising adding pollutant absorbing substances to the encapsulation material.

11. The method of claim 7, further comprising adding stabilizing structures to the encapsulation material.

12. The method of claim 7, wherein combining the vegetation with the encapsulating material forms a primary encapsulation.

13. The method of claim 12, wherein the primary encapsulation is a surrounded by one or more additional layers of encapsulation that form a secondary encapsulation.

14. The method of claim 13, wherein a material for the secondary encapsulation is different from the material for the primary encapsulation.

15. The method of claim 7, further comprising directing light selected to irradiate the vegetation to the cultivation environment.

16. The method of claim 7, wherein the cultivation environment contains water as a cultivation medium.

17. The method of claim 7, further comprising sterilizing the vegetation.

18. The method of claim 7, further comprising pressing the vegetation.
